(19) United States
(12) Patent Application Publication
(10) Pub. No.: US 2026/0021094 A1
(43) Pub. Date: Jan. 22, 2026

(54) COMPOSITION FOR IMPROVING THE EFFICACY OF HAIR GROWTH ACTIVE SUBSTANCE AND PREPARATION METHOD THEREOF

(71) Applicant: ARTIVILA BIOPHARMA, Wuxi, Jiangsu (CN)

(72) Inventors: Zhendong Zhu, Wuxi, Jiangsu (CN); Jian Tang, Wuxi, Jiangsu (CN); Deqiang Niu, Wuxi, Jiangsu (CN); Wen Long, Wuxi, Jiangsu (CN); Qi Yang, Wuxi, Jiangsu (CN)

(21) Appl. No.: 18/993,077

(22) PCT Filed: Jun. 30, 2023
(86) PCT No.: PCT/CN2023/105197

§ 371 Date: Jan. 10, 2025

(30) Foreign Application Priority Data
Jul. 12, 2022 (CN) 202210817175.6

(51) Int. Cl. A61K 31/506; A61K 31/58; A61K 47/10; A61K 47/12; A61K 47/14; A61K 47/46; A61P 17/14

(52) U.S. Cl. CPC A61K 31/506; A61K 31/58; A61K 47/10; A61K 47/12; A61K 47/14; A61K 47/46; A61P 17/14

(57) ABSTRACT

A composition for improving the efficacy of a hair growth active substance and a preparation method therefor, wherein the composition comprises a stabilizer, a pharmaceutically acceptable carrier, and lipophilic particles suspended in the carrier, and the lipophilic particles comprise the hair growth active substance, a penetration enhancer, and a surfactant; and the preparation method comprises: heating and dissolving the hair growth active substance and the pharmaceutically acceptable carrier, adding water, the penetration enhancer, and the stabilizer, heating and stirring to obtain a mixture heating and melting the remaining penetration enhancer and the surfactant and mixing same with the mixture.

### FIELD

The invention relates to the technical field of local external medicaments, particularly a composition for improving the efficacy of hair growth active substances and preparation methods.

### BACKGROUND

Although hair loss or baldness is considered a normal aging phenomenon in the elderly and will not receive special attention, serious hair loss occurs in young and middle-aged men and women, and symptoms such as baldness can cause both psychological and spiritual deep and continuous torture for individuals. Various factors lead to hair loss, such as mental stress, excessive stress in life; disease factors; and medication factors that can cause severe hair loss. One of the hair loss factors with a higher incidence is hereditary hair loss, also known as androgenetic alopecia. The patient's hair follicles and sebaceous glands secrete too much androgen, which strongly stimulates the hair follicle cells to shrink the hair follicles, thus preventing the normal metabolism and growth of hair.

For many years there has been a strong practical need for therapeutic products to promote hair growth or prevent hair loss among groups of consumers or patients. For example, the consumer market is filled with a large number of various plants, herbal hair growth, and hair care products, of which ginger is a kind of hair growth product with high influence. However, only minoxidil, finasteride, and so on have been proven effective for hair growth by rigorous clinical studies. Among them, minoxidil was developed as an external preparation, and finasteride produced an effect by oral administration. Due to the convenience and low risk of external preparations, minoxidil-type external hair growth products have gained a higher market share. Minoxidil dosage forms approved in China include tincture, liniment, spray, gel, and foam dosage forms abroad. Patent CN1154463C, for example, provides a liquid sustained release hair growth preparation of minoxidil that is used after 20-fold dilution with pure water. Patent CN100577168C provides a minoxidil traditional cream preparation for treating alopecia, with a high content of stearic acid, vaseline, liquid paraffin, glycerol, and the like in the oil phase, and the use convenience of the cream on hair is reduced, in particular, the emollient property is poor. The greasy feeling is heavy, which is not favorable for patients to use for a long time. Patent CN106659647B provides a liquid vesicle containing minoxidil suspended in a liquid carrier.

The market leader is minoxidil tincture, containing a large amount of propylene glycol and ethanol, which has strong skin irritation and a high incidence of skin allergy and dandruff. Liniments were similar to tinctures, except that there were some differences in the use method, and they were liquid preparations, with poor retention on the skin and low bioavailability of the active ingredients. The spray is prepared by adding propellant based on tincture and has low accuracy. The gel dosage form added the gel component based on tincture, and it was expected to improve drug utilization by increasing the retention time at the administration site, but the contained skin-stimulating component was not fundamentally improved.

### SUMMARY

The technical problem to be solved by the present invention is that, in response to the prior art, there is room for further improvement in the utilization or efficacy of hair growth drugs of tinctures, which are the most prevalent on the market, compositions, and methods of preparation thereof are provided for improving the efficacy of hair growth active substances.

The technical scheme adopted by the invention for solving the technical problems is as follows:

### The First Technical Scheme:

The invention relates to a composition for improving the efficacy of hair growth active substance, comprising the following materials in parts by weight: 0.5-5 parts of a stabilizer, 20-80 parts of a pharmaceutically acceptable carrier, and 3.1-82 parts of lipophilic particles suspended in the carrier; the lipophilic particles have a core-shell structure or homogeneous entities, and the lipophilic particles comprise 0.5-10 parts of the hair growth active substance, 1.6-52 parts of the a penetration enhancer, and 1-20 parts of a surfactant; the penetration enhancer comprises the following materials in parts by weight: 0.5-25 parts of a C₁₀-C₂₅ alcohol, 0.5-5 parts of a pharmaceutically acceptable grease, 0.5-12 parts of a C₁₂-C₂₀ alkanoic acid and 0.1-10 parts of a ginger extract; the hair growth active substance is at least one of minoxidil or pharmaceutically acceptable salt thereof and finasteride.

Preferably, the stabilizer is at least one of polyethylene glycol, C₂-C₆ monohydric or polyhydric alcohol, sodium dodecyl sulfate, disodium EDTA, Tween 80, Tween 60, Tween 20, water-soluble cellulose or derivatives thereof, carbomer and xanthan gum.

Preferably, the penetration enhancer comprises 0.5-15 parts by weight of a C₁₄-C₂₀ alcohol.

Preferably, the pharmaceutically acceptable grease is at least one of lanolin, white vaseline, and yellow vaseline.

Preferably, the alkanoic acid in the penetration enhancer is dodecanoic acid, tetradecanoic acid, hexadecanoic acid, heptadecanoic acid, octadecanoic acid or eicosanoic acid.

Preferably, the surfactant is a C₃-C₆ alcohol ester of fatty acid; wherein the fatty acid comprises a C₁₀-C₂₂ saturated fatty acid or unsaturated fatty acid; the C₃-C₆ alcohol ester is a monoester or polyester, the weight parts of C₃-C₆ alcohol ester are 1-12 parts.

Preferably, the pharmaceutically acceptable carrier is at least one of water and C₂-C₆ alcohols, wherein the C₂-C₆ alcohols are monohydric alcohols or polyhydric alcohols.

The preparation method of a composition for improving the efficacy of hair growth active substance comprises the following steps of, the raw material in parts by weight:
S1, heating and dissolving 0.5-10 parts of hair growth active substance and 20-80 parts of the pharmaceutically acceptable carrier at 60-100° C. to obtain a first mixture, wherein the hair growth active substance is at least one of minoxidil or pharmaceutically acceptable salt thereof and finasteride;
S2, adding 20-60 parts of water, 0.1-10 parts of a penetration enhancer ginger extract and, 0.5-5 parts of a stabilizer into the first mixture in the S1 step, heating and stirring at the temperature of 60-100° C. and the rotating speed of 100-500 rpm to obtain a clarified second mixture;
S3, heating and melting 1.5-42 parts of other penetration enhancer and 1-20 parts of a surfactant at the temperature of 60-100° C. to obtain a third mixture, wherein the other penetration enhancer comprises the following materials in parts by weight: 0.5-25 parts of a C₁₀-C₂₅ alcohol, 0.5-5 parts of a pharmaceutically acceptable grease and 0.5-12 parts of C₁₂-C₂₀ alkanoic acid;
S4, adding the second mixture obtained in the S2 step into the third mixture obtained in the S3 step within 5 minutes, stirring for 5-15 minutes at the rotating speed of 100-500 rpm, and then homogenizing for 10-20 minutes at the rotating speed of 4000-10000 rpm to obtain the composition that promotes hair growth efficacy with the active substance.

### The Second Technical Scheme:

The invention relates to a composition for improving the efficacy of hair growth active substance, comprising the following materials in parts by weight: 0.5-10 parts of hair growth active substance dispersed in a carrier, wherein the carrier comprises 15.1-100 parts of a penetration enhancer, 5-20 parts of a surfactant and 15-30 parts of solvent; the penetration enhancer comprises the following materials in parts by weight: 5-25 parts of a C₁₀-C₂₅ alcohol, 5-50 parts of a pharmaceutically acceptable grease, 5-15 parts of a C₁₂-C₂₀ alkanoic acid and 0.1-10 parts of a ginger extract; the hair growth active substance is at least one of minoxidil or pharmaceutically acceptable salt thereof and finasteride.

Preferably, the pharmaceutically acceptable grease is at least one of lanolin, white vaseline, and yellow vaseline.

Preferably, the alkanoic acid in the penetration enhancer is dodecanoic acid, tetradecanoic acid, hexadecanoic acid, heptadecanoic acid, octadecanoic acid or eicosanoic acid.

Preferably, the surfactant is a C₃-C₆ alcohol ester of fatty acid; wherein the fatty acid comprises a C₁₀-C₂₂ saturated fatty acid or unsaturated fatty acid; the C₃-C₆ alcohol ester is a monoester or polyester, the weight parts of the C₃-C₆ alcohol ester are 5-12 parts.

Preferably, the solvent is a C₂-C₆ monohydric alcohol or a C₂-C₆ polyhydric alcohol.

The preparation method of a composition for improving the efficacy of hair growth active substance according to the second technical scheme comprises the following steps of, the raw material in parts by weight:
S1, heating and dissolving 0.5-10 parts of hair growth active substance and 15-30 parts of solvent at 60-100° C. to obtain a first mixture, wherein the hair growth active substance is at least one of minoxidil or pharmaceutically acceptable salt thereof and finasteride;
S2, weigh a carrier, wherein the carrier comprises 15.1-100 parts of a penetration enhancer, 5-20 parts of a surfactant, and 15-30 parts of a solvent; heating and melting 15.1-100 parts of a penetration enhancer and 5-20 parts of a surfactant at the temperature of 60-100° C. to obtain a second mixture, wherein the penetration enhancer comprises the following materials in parts by weight: 5-25 parts of a C₁₀-C₂₅ alcohol, 5-50 parts of a pharmaceutically acceptable grease, 5-15 parts of a C₁₂-C₂₀ alkanoic acid and 0.1-10 parts of a ginger extract;
S3, adding the first mixture obtained in the S1 step into the second mixture obtained in the S2 step within 5 minutes, stirring for 5-15 minutes at the rotating speed of 100-500 rpm, and then homogenizing for 10-20 minutes at the rotating speed of 4000-10000 rpm to obtain the composition that promotes hair growth efficacy with the active substance.

The invention has the beneficial effects that:

According to the composition for improving the efficacy of hair growth active substance, compared with the side effects existing in various dosage form products on the market, the corresponding side effects are remarkably reduced or even eliminated, the lipophilic particles containing the hair growth active substance in the composition have good permeability on skin, the efficacy of the hair growth active substance is remarkably improved, and the hair growth is promoted more efficiently.

The preparation method of the composition for improving the efficacy of hair growth active substance has low production cost and high finished product yield.

The composition for improving the efficacy of hair growth active substance of the present invention also remarkably improves the efficacy of the hair growth active substance and more efficiently promotes hair generation.

The preparation method of the composition for improving the efficacy of hair growth active substance has the advantages of simple and convenient operation, low production cost, suitability for large-scale production, and economy.

### BRIEF DESCRIPTION OF THE DRAWINGS

FIG. 1 is a photograph under an optical microscope of lipophilic particles in the composition of example 1 of the present invention;

FIG. 2 is the results of an in vitro transdermal penetration test of example 1 of the present invention and a commercial tincture;

FIG. 3 is the results of an 8 hours in vitro transdermal permeation test of example 1 of the present invention and a commercial tincture;

FIG. 4 is the results of an in vitro transdermal permeation test of example 8 of the present invention and a commercial tincture;

FIG. 5 is the results of an 8 hours in vitro transdermal permeation test of example 8 of the present invention and a commercial tincture; and

FIG. 6 is a graph showing the results of hair growth tests in animals according to example 1, example 2, and the commercial tincture of the present invention.

### DETAILED DESCRIPTION

To have a clearer understanding of the technical features, objects, and effects of the present invention, the present invention will be further described in detail below with reference to the examples, which are merely for explaining the present invention and are not intended to limit the scope of the present invention.

The first embodiment of the present invention is:

The invention relates to a composition for improving the efficacy of hair growth active substance, including the following materials in parts by weight: 0.5-5 parts of a stabilizer, 20-80 parts of a pharmaceutically acceptable carrier, and 3.1-82 parts of lipophilic particles suspended in the carrier; the lipophilic particles have a core-shell structure or a homogeneous entity, and the particle size is 0.5-5 μm; the lipophilic particles comprise 0.5-10 parts of hair growth active substance, 1.6-52 parts of a penetration enhancer, and 1-20 parts of a surfactant; the penetration enhancer comprises the following materials in parts by weight: 0.5-25 parts of a C₁₀-C₂₅ alcohol, 0.5-5 parts of a pharmaceutically acceptable grease, 0.5-12 parts of a C₁₂-C₂₀ alkanoic acid and 0.1-10 parts of a ginger extract; the hair growth active substance is at least one of minoxidil or pharmaceutically acceptable salt thereof and finasteride.

Wherein the stabilizer is at least one of polyethylene glycol, C₂-C₆ monohydric or polyhydric alcohol, sodium dodecyl sulfate (SDS), disodium EDTA, Tween 80, Tween 60, Tween 20, water-soluble cellulose or derivatives thereof, carbomer and xanthan gum. According to the invention, the lipophilic particles can be stably dispersed in a carrier by adding a stabilizer into the composition of the hair growth active substance.

The alcohol in the penetration enhancer is a C₁₄-C₂₀ alcohol, preferably 0.5-15 parts. The pharmaceutically acceptable grease is at least one of lanolin, white vaseline, and yellow vaseline. The alkanoic acid in the penetration enhancer is dodecanoic acid, tetradecanoic acid, hexadecanoic acid, heptadecanoic acid, octadecanoic acid or eicosanoic acid. The penetration enhancer can improve the permeability of the hair growth active substance composition of the present invention to the skin and promoting adequate absorption of drugs.

The surfactant is a C₃-C₆ alcohol ester of fatty acid, wherein the fatty acid comprises a C₁₀-C₂₂ saturated fatty acid or unsaturated fatty acid, and the alcohol ester comprises monoester or polyester; the surfactant is preferably 1.5-12 parts by weight.

The pharmaceutically acceptable carrier is at least one of water, C₂-C₆ alcohols, wherein the C₂-C₆ alcohols are monohydric alcohols or polyhydric alcohols.

The invention also provides a preparation method of the composition for improving the efficacy of hair growth active substance, including the following steps:
S1, heating and dissolving 0.5-10 parts of hair growth active substance and 20-80 parts of the pharmaceutically acceptable carrier at 60-100° C. to obtain a first mixture, wherein the hair growth active substance is at least one of minoxidil or pharmaceutically acceptable salt thereof and finasteride; preferably, the pharmaceutically acceptable carrier is at least one of water and C₂-C₆ alcohols, wherein the C₂-C₆ alcohols are monohydric alcohols or polyhydric alcohols;
S2, adding 20-60 parts of water, 0.1-10 parts of a penetration enhancer ginger extract and, 0.5-5 parts of a stabilizer into the first mixture in the S1 step, heating and stirring at the temperature of 60-100° C. and the rotating speed of 100-500 rpm to obtain a clarified second mixture. Preferably, the stabilizer is at least one of polyethylene glycol, C₂-C₆ monohydric or polyhydric alcohol, sodium dodecyl sulfate, disodium EDTA, Tween 80, Tween 60, Tween 20, water-soluble cellulose or derivatives thereof, carbomer and xanthan gum.

S3, heating and melting 1.5-42 parts of other penetration enhancer and 1-20 parts of a surfactant at the temperature of 60-100° C. to obtain a third mixture, wherein the other penetration enhancer comprises the following materials in parts by weight: 0.5-25 parts of a C₁₀-C₂₅ alcohol, 0.5-5 parts of a pharmaceutically acceptable grease and 0.5-12 parts of a C₁₂-C₂₀ alkanoic acid;

S4, adding the second mixture obtained in the S2 step into the third mixture obtained in the S3 step within 5 minutes, stirring for 5-15 minutes at the rotating speed of 100-500 rpm, and then homogenizing for 10-20 minutes at the rotating speed of 4000-10000 rpm to obtain the composition that promotes hair growth efficacy with the active substance.

The composition made in this embodiment is a homogeneous liquid emulsion.

### The Second Embodiment is

The invention relates to a composition for improving the efficacy of hair growth active substance, including the following materials in parts by weight: 0.5-10 parts of hair growth active substance dispersed in a carrier, wherein the carrier comprises 15.1-100 parts of a penetration enhancer, 5-20 parts of a surfactant and 15-30 parts of solvent; the penetration enhancer comprises the following materials in parts by weight: 5-25 parts of a C₁₀-C₂₅ alcohol, 5-50 parts of a pharmaceutically acceptable grease, 5-15 parts of a C₁₂-C₂₀ alkanoic acid, and 0.1-10 parts of a ginger extract; the hair growth active substance is at least one of minoxidil or pharmaceutically acceptable salt thereof and finasteride.

The pharmaceutically acceptable grease is at least one of lanolin, white vaseline, and yellow vaseline.

The alkanoic acid in the penetration enhancer is dodecanoic acid, tetradecanoic acid, hexadecanoic acid, heptadecanoic acid, octadecanoic acid or eicosanoic acid.

The surfactant is a C₃-C₆ alcohol ester of fatty acid, wherein the fatty acid comprises a C₁₀-C₂₂ saturated fatty acid or unsaturated fatty acid, and the C₃-C₆ alcohol ester is a monoester or polyester. The surfactant is preferably 5-12 parts by weight.

The solvent is a C₂-C₆ monohydric alcohol or a C₂-C₆ polyhydric alcohol.

The invention also provides a preparation method of the composition for improving the efficacy of hair growth active substance according to the second embodiment, including the following steps of, the raw material in parts by weight:

S1, heating and dissolving 0.5-10 parts of hair growth active substance and 15-30 parts of solvent at 60-100° C. to obtain a first mixture, wherein the hair growth active substance is at least one of minoxidil or pharmaceutically acceptable salt thereof and finasteride; preferably the solvent is a C₂-C₆ monohydric alcohol or a C₂-C₆ polyhydric alcohol.

S2, weigh a carrier, wherein the carry comprises 15.1-100 parts of a penetration enhancer, 5-20 parts of a surfactant, and 15-30 parts of a solvent; heating and melting 15.1-100 parts of the penetration enhancer and 5-20 parts of the surfactant at the temperature of 60-100° C. to obtain a second mixture, wherein the penetration enhancer comprises the following materials in parts by weight: 5-25 parts of a C₁₀-C₂₅ alcohol, 5-50 parts of a pharmaceutically acceptable grease, 5-15 parts of a C₁₂-C₂₀ alkanoic acid, and 0.1-10 parts of a ginger extract; preferably the surfactant is a C₃-C₆ alcohol ester of fatty acid; wherein the fatty acid comprises a C₁₀-C₂₂ saturated fatty acid or unsaturated fatty acid; the C₃-C₆ alcohol ester is a monoester or polyester, the weight parts of the C₃-C₆ alcohol ester are 5-12 parts.

S3, adding the first mixture obtained in the S1 step into the second mixture obtained in the S2 step within 5 minutes, stirring for 5-15 minutes at the rotating speed of 100-500 rpm, and then homogenizing for 10-20 minutes at the rotating speed of 4000-10000 rpm to obtain the composition that promotes hair growth efficacy with the active substance.

The composition made in this embodiment is a homogeneously mixed solid or semi-solid paste.

The composition for improving the efficacy of hair growth active substance is an emulsion with a certain flow, or a semi-flow or non-flow paste, and compared with the side effects existing in various dosage forms of products on the market, the composition remarkably reduces or even eliminates the corresponding side effects; and the composition has good permeability on skin, remarkably improves the efficacy of the hair growth active substance, and promotes hair growth more efficiently.

The preparation method of the composition for improving the efficacy of hair growth active substance has a simple and convenient preparation process and low production cost, is suitable for large-scale production, and is economical.

The following is illustrated by specific examples, in which examples 1 to 7 are used for preparing emulsions and examples 8 to 12 are used for preparing semi-solid or solid pastes:

### Example 1

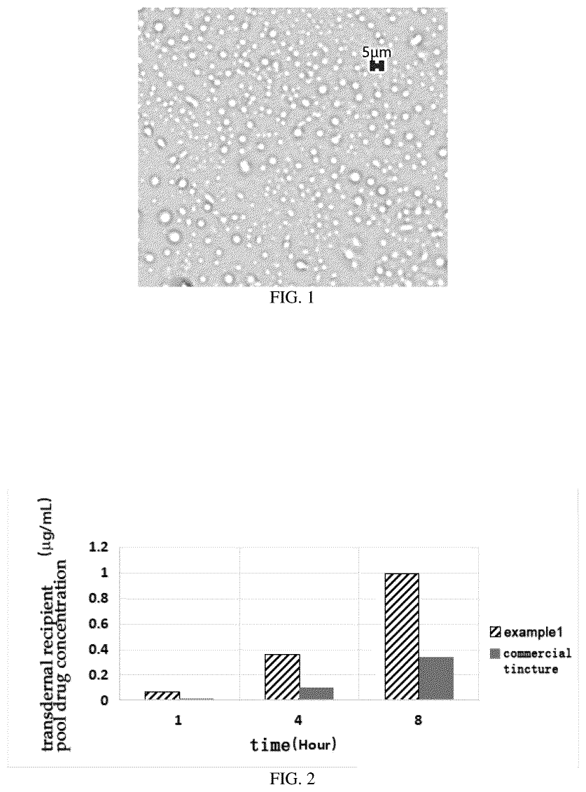

A composition for improving the efficacy of hair growth active substance comprises the following materials in parts by weight: 3 parts of a stabilizer (polyethylene glycol, SDS, Tween 80 and disodium EDTA in a mass ratio of 1:1:1:1), 35 parts of a pharmaceutically acceptable carrier (glycerol), and 40.5 parts of lipophilic particles suspended in the carrier; the lipophilic particles have a core-shell structure and a particle size of 2.5 μm; the lipophilic particles comprise 5 parts of hair growth active substance (minoxidil), 23.5 parts of a penetration enhancer (including 12 parts of cetyl alcohol, 3 parts of a ginger extract, 2.5 parts of lanolin, and 6 parts of hexadecanoic acid), and 12 parts of a surfactant (glycerol monostearate). A photograph of the lipophilic particles of this example under an optical microscope is shown in FIG. 1, in which the lipophilic particles are homogeneously dispersed and suspended.

The invention relates to a preparation method of a composition for improving the efficacy of hair growth active substance, including the following steps of, the raw material in parts by weight:
S1, heating and dissolving 5 parts of minoxidil and 35 parts of glycerol at 80° C. to obtain a first mixture;
S2, adding 30 parts of water, 3 parts of a ginger extract and 3 parts of a stabilizer (polyethylene glycol, SDS, Tween 80 and disodium EDTA in a mass ratio of 1:1:1:1) into the first mixture in the S1 step, heating and stirring at 80° C. at a rotating speed of 300 rpm to obtain a clarified second mixture;
S3, heating and melting 12 parts of cetyl alcohol, 2.5 parts of lanolin, 6 parts of hexadecanoic acid and 12 parts of glycerol monostearate at 80° C. to obtain a third mixture;
S4, adding the second mixture of S2 into the third mixture obtained in the S3 step within 3 minutes while stirring at a rotation speed of 300 rpm for 10 minutes, and then homogenizing at a rotation speed of 7000 rpm for 15 minutes to obtain a composition having hair growth active substance.

### Example 2

A composition for improving the efficacy of hair growth active substance comprises the following materials in parts by weight: 5 parts of a stabilizer (ethanol, SDS, Tween 60, and xanthan gum in a mass ratio of 2:2:1:1), 20 parts of the pharmaceutically acceptable carrier (glycerol), and 15 parts of lipophilic particles suspended in the carrier; the lipophilic particles have a core-shell structure and a particle size of 2.5 μm; the lipophilic particles comprise 5 parts of hair growth active substance (minoxidil), 8 parts of a penetration enhancer (including 2 parts of cetyl alcohol, 3 parts of a ginger extract, 2 parts of lanolin and 1 part of hexadecanoic acid), and 2 parts of a surfactant (glycerol monostearate).

The invention relates to a preparation method of a composition for improving the efficacy of hair growth active substance, including the following steps of, the raw material in parts by weight:
S1, heating and dissolving 5 parts of minoxidil and 20 parts of glycerol at 80° C. to obtain a first mixture;
S2, adding 50 parts of water, 3 parts of a ginger extract and 5 parts of a stabilizer (ethanol, SDS, Tween 60 and xanthan gum in a mass ratio of 2:2:1:1) into the first mixture in the S1 step, heating and stirring at 80° C. at a rotating speed of 300 rpm to obtain a clarified second mixture;
S3, heating and melting 2 parts of cetyl alcohol, 2 parts of lanolin, 1 part of hexadecanoic acid, and 2 parts of glycerol monostearate at 80° C. to obtain a third mixture;
S4, adding the second mixture of S2 into the third mixture obtained in the S3 step within 3 minutes while stirring at a rotation speed of 300 rpm for 10 minutes, and then homogenizing at a rotation speed of 7000 rpm for 15 minutes to obtain a composition having hair growth active substance.

### Example 3

A composition for improving the efficacy of hair growth active substance comprises the following materials in parts by weight: 0.5 parts of a stabilizer (glycerol, disodium EDTA, and water-soluble cellulose in a mass ratio of 1:1:1), 20 parts of the pharmaceutically acceptable carrier (ethanol), and 14.5 parts of lipophilic particles suspended in the carrier; the lipophilic particles are homogeneous entities and have a particle size of 0.5 μm; the lipophilic particles comprise 2 parts of hair growth active substance (finasteride), 7.5 parts of a penetration enhancer (including 5 parts of decanol, 0.5 parts of white vaseline, 0.5 parts of dodecanoic acid, and 1.5 parts of a ginger extract), and 5 parts of a surfactant (n-propyl decanoate).

The invention relates to a preparation method of a composition for improving the efficacy of hair growth active substance, including the following steps of, the raw material in parts by weight:
S1, heating and dissolving 2 parts of finasteride and 20 parts of ethanol at 60° C. to obtain a first mixture;
S2, adding 20 parts of water, 1.5 parts of a ginger extract, and 0.5 parts of a stabilizer (glycerol, disodium EDTA, and water-soluble cellulose in a mass ratio of 1:1:1) into the first mixture in the S1 step, heating and stirring at 60° C. at a rotating speed of 500 rpm to obtain a clarified second mixture;
S3, heating and melting 5 parts of decanol, 0.5 parts of white vaseline, 0.5 parts of dodecanoic acid, and 5 parts of n-propyl decanoate at 60° C. to obtain a third mixture;
S4, adding the second mixture of S2 into the third mixture obtained in the S3 step within 1 minute while stirring at 100 rpm for 15 minutes, and then homogenizing at 4000 rpm for 20 minutes to obtain a composition having hair growth active substance.

### Example 4

A composition for improving the efficacy of hair growth active substance comprises the following materials in parts by weight: 5 parts of a stabilizer (Tween 20, polyanionic cellulose, and carbomer in a mass ratio of 2:1:1), 50 parts of the pharmaceutically acceptable carrier (hexanol), and 70 parts of lipophilic particles suspended in the carrier; the lipophilic particles are homogeneous entities and have a particle size of 5 μm; the lipophilic particles comprise 8 parts of hair growth active substance (minoxidil sulfate), 42 parts of a penetration enhancer (including 23.5 parts of peanut alcohol, 5 parts of yellow vaseline, 12 parts of eicosanoic acid, and 1.5 parts of a ginger extract), and 20 parts of a surfactant (butyl laurate).

The invention relates to a preparation method of a composition for improving the efficacy of hair growth active substance, including the following steps of, the raw material in parts by weight:
S1, heating and dissolving 8 parts of hair growth active substance (minoxidil sulfate) and 50 parts of hexanol at 100° C. to obtain a first mixture;
S2, adding 45 parts of water, 1.5 parts of a ginger extract, and 5 parts of a stabilizer (Tween 20, polyanionic cellulose, and carbomer in a mass ratio of 2:1:1) into the first mixture in the S1 step, heating and stirring at 100° C. at a rotating speed of 100 rpm to obtain a clarified second mixture;
S3, heating and melting 23.5 parts of peanut alcohol, 5 parts of yellow vaseline, 12 parts of eicosanoic acid, and 20 parts of butyl laurate at 100° C. to obtain a third mixture;
S4, adding the second mixture of S2 into the third mixture obtained in the S3 step within 5 minutes while stirring at 500 rpm for 5 minutes, and then homogenizing at 10000 rpm for 10 minutes to obtain a composition having hair growth active substance.

### Example 5

A composition for improving the efficacy of hair growth active substance comprises the following materials in parts by weight: 0.5 parts of a stabilizer (glycerol), 20 parts of the pharmaceutically acceptable carrier (ethanol), and 3.1 parts of lipophilic particles suspended in the carrier; the lipophilic particles are homogeneous entities and have a particle size of 0.5 μm; the lipophilic particles comprise 0.5 parts of hair growth active substance (finasteride), 1.6 parts of a penetration enhancer (including 0.5 parts of decanol, 0.5 parts of white vaseline, 0.5 parts of dodecanoic acid, and 0.1 parts of a ginger extract), and 1 part of a surfactant (n-propyl decanoate).

The invention relates to a preparation method of a composition for improving the efficacy of hair growth active substance, including the following steps of, the raw material in parts by weight:
S1, heating and dissolving 0.5 parts of finasteride and 20 parts of ethanol at 60° C. to obtain a first mixture;
S2, adding 20 parts of water, 0.1 parts of a ginger extract, and 0.5 parts of a stabilizer (glycerol) into the first mixture of S1, heating and stirring at 60° C. at a rotation speed of 500 rpm to obtain a clarified second mixture;
S3, heating and melting 0.5 parts of decanol, 0.5 parts of white vaseline, 0.5 parts of dodecanoic acid, and 1 part of n-propyl decanoate at 60° C. to obtain a third mixture;
S4, adding the second mixture of S2 into the third mixture obtained in the S3 step within 1 minute while stirring at 100 rpm for 15 minutes, and then homogenizing at 4000 rpm for 20 minutes to obtain a composition having hair growth active substance.

### Example 6

A composition for improving the efficacy of hair growth active substance comprises the following materials in parts by weight: 5 parts of a stabilizer (Tween 20, polyanionic cellulose, and carbomer in a mass ratio of 2:1:0.5), 80 parts of the pharmaceutically acceptable carrier (hexanol), and 67 parts of lipophilic particles suspended in the carrier; the lipophilic particles are homogeneous entities and have a particle size of 5 μm; the lipophilic particles comprise 10 parts of hair growth active substance (minoxidil sulfate), 42 parts of a penetration enhancer (including 15 parts of peanut alcohol, 5 parts of yellow vaseline, 12 parts of eicosanoic acid, and 10 parts of a ginger extract), and 15 parts of a surfactant (butyl laurate).

The invention relates to a preparation method of a composition for improving the efficacy of hair growth active substance, including the following steps of, the raw material in parts by weight:
S1, heating and dissolving 10 parts of hair growth active substance (minoxidil sulfate) and 80 parts of hexanol at 90° C. to obtain a first mixture;
S2, adding 60 parts of water, 10 parts of a ginger extract, and 5 parts of a stabilizer (Tween 20, polyanionic cellulose, and carbomer in a mass ratio of 2:1:0.5) into the first mixture in the S1 step, heating and stirring at 90° C. at a rotating speed of 200 rpm to obtain a clarified second mixture;
S3, heating and melting 15 parts of peanut alcohol, 5 parts of yellow vaseline, 12 parts of eicosanoic acid and 15 parts of butyl laurate at 90° C. to obtain a third mixture;
S4, adding the second mixture of S2 into the third mixture obtained in the S3 step within 4 minutes while stirring at 400 rpm for 8 minutes, and then homogenizing at 8000 rpm for 12 minutes to obtain a composition having hair growth active substance.

### Example 7

A composition for improving the efficacy of hair growth active substance comprises the following materials in parts by weight: 5 parts of a stabilizer (disodium EDTA, hexanol, and Tween 80 in a mass ratio of 1:1:1:1), 80 parts of the pharmaceutically acceptable carrier (hexanol), and 82 parts of lipophilic particles suspended in the carrier; the lipophilic particles are homogeneous entities and have a particle size of 5 μm; the lipophilic particles comprise 10 parts of hair growth active substance (minoxidil sulfate and finasteride in a mass ratio of 1:1), 52 parts of a penetration enhancer (including 25 parts of peanut alcohol, 5 parts of yellow vaseline, 12 parts of eicosanoic acid, and 10 parts of a ginger extract), and 20 parts of a surfactant (butyl laurate).

The invention relates to a preparation method of a composition for improving the efficacy of hair growth active substance, including the following steps of, the raw material in parts by weight:
S1, heating and dissolving 10 parts of hair growth active substance (minoxidil sulfate and finasteride in a mass ratio of 1:1) and 80 parts of hexanol at 100° C. to obtain a first mixture;
S2, adding 60 parts of water, 10 parts of a ginger extract, and 5 parts of a stabilizer (disodium EDTA, hexanol, and Tween 80 in a mass ratio of 1:1:1:1) into the first mixture in the S1 step, heating and stirring at 100° C. at a rotating speed of 100 rpm to obtain a clarified second mixture;
S3, heating and melting 25 parts of peanut alcohol, 5 parts of yellow vaseline, 12 parts of eicosanoic acid, and 20 parts of butyl laurate at 100° C. to obtain a third mixture;
S4, adding the second mixture of S2 into the third mixture obtained in the S3 step within 5 minutes while stirring at 500 rpm for 5 minutes, and then homogenizing at 10000 rpm for 10 minutes to obtain a composition having hair growth active substance.

It will be appreciated that in the above Examples and alternatives thereof, the stabilizer may be replaced with at least one of polyethylene glycol, C₂-C₆ monohydric or polyhydric alcohol, sodium dodecyl sulfate (SDS), disodium EDTA, Tween 80, Tween 60, Tween 20, water-soluble cellulose or derivatives thereof, carbomer, xanthan gum; the hair growth active substance may be replaced with at least one of minoxidil or a pharmaceutically acceptable salt thereof, finasteride; the alcohol in the penetration enhancer may be replaced with a C₁₀-C₂₅ alcohol; the pharmaceutically acceptable grease can be replaced with at least one of lanolin, white vaseline, and yellow vaseline; the alkanoic acid in the penetration enhancer may be replaced with tetradecanoic acid, heptadecanoic acid or octadecanoic acid; the surfactant may be replaced by C₃-C₆ alcohol esters of C₁₀-C₂₂ fatty acids; the carrier may be replaced with at least one of C₂-C₆ monohydric alcohol or C₂-C₆ polyhydric alcohol. The replaced composition also has the technical effects of the present invention.

### Example 8

A composition for improving the efficacy of hair growth active substance comprises the following materials in parts by weight: 5 parts of hair growth active substance (minoxidil) dispersed in a carrier, wherein the carrier comprises 49.5 parts of a penetration enhancer (12 parts of a heptadecanol, 30 parts of a white vaseline, 1.5 parts of a ginger extract, and 6 parts of a heptadecanoic acid), 13 parts of a surfactant (glycerol monolaurate), and 22 parts of a solvent (butanol).

The invention relates to a preparation method of a composition for improving the efficacy of hair growth active substance, including the following steps of, the raw material in parts by weight:
S1, heating and dissolving 5 parts of minoxidil and 22 parts of butanol at 80° C. to obtain a first mixture;
S2, heating and melting 12 parts of heptadecanol, 30 parts of white vaseline, 1.5 parts of a ginger extract, 6 parts of heptadecanoic acid, and 13 parts of glycerol monolaurate at 80° C. to obtain a second mixture;
S3, adding the first mixture obtained in the S1 step into the second mixture obtained in the S2 step within 3 minutes while stirring at a rotation speed of 300 rpm for 10 minutes, and then homogenizing at a rotation speed of 7000 rpm for 15 minutes to obtain a composition having hair growth active substance.

### Example 9

A composition for improving the efficacy of hair growth active substance comprises the following materials in parts by weight: 0.5 parts of hair growth active substance (finasteride) dispersed in a carrier, wherein the carrier comprises 15.1 parts of a penetration enhancer (5 parts of lauryl alcohol, 5 parts of yellow vaseline, 0.1 parts of a ginger extract, and 5 parts of dodecanoic acid), 5 parts of a surfactant (glycerol monooleate) and 15 parts of solvent (glycerol).

The invention relates to a preparation method of a composition for improving the efficacy of hair growth active substance, including the following steps of, the raw material in parts by weight:
S1, heating and dissolving 0.5 parts of hair growth active substance (finasteride) and 15 parts of glycerol at 60° C. to obtain a first mixture;
S2, heating and melting 5 parts of dodecanol, 5 parts of yellow vaseline, 0.1 parts of a ginger extract, 5 parts of dodecanoic acid, and 5 parts of glycerol monooleate at 60° C. to obtain a second mixture;
S3, adding the first mixture obtained in the S1 step into the second mixture obtained in the S2 step within 1 minute while stirring at 100 rpm for 15 minutes, and then homogenizing at 4000 rpm for 20 minutes to obtain a composition having hair growth active substance.

### Example 10

A composition for improving the efficacy of hair growth active substance comprises the following materials in parts by weight: 8 parts of hair growth active substance (minoxidil and finasteride in a mass ratio of 2:1) dispersed in a carrier, wherein the carrier comprises 48 parts of a penetration enhancer (25 parts of a behenyl alcohol, 5 parts of a lanolin, 3 parts of a ginger extract and 15 parts of a eicosanoic acid), 20 parts of a surfactant (butyl palmitate) and 30 parts of a solvent (pentanol).

The invention relates to a preparation method of a composition for improving the efficacy of hair growth active substance, including the following steps of, the raw material in parts by weight:
S1, heating and dissolving 8 parts of hair growth active substances (minoxidil and finasteride in a mass ratio of 2:1) and 30 parts of pentanol at 100° C. to obtain a first mixture;
S2, heating and melting 25 parts of behenyl alcohol, 5 parts of lanolin, 3 parts of a ginger extract, 15 parts of eicosanoic acid, and 20 parts of butyl palmitate at 100° C. to obtain a second mixture;
S3, adding the first mixture obtained in the S1 step into the second mixture obtained in the S2 step within 5 minutes while stirring for 5 minutes at a rotation speed of 500 rpm, and then homogenizing for 10 minutes at a rotation speed of 10000 rpm to obtain a composition having hair growth active substance.

### Example 11

A composition for improving the efficacy of hair growth active substance comprises the following materials in parts by weight: 10 parts of hair growth active substance (minoxidil sulfate) dispersed in a carrier, wherein the carrier comprises 93 parts of a penetration enhancer (25 parts of behenyl alcohol, 50 parts of lanolin, 3 parts of a ginger extract, and 15 parts of eicosanoic acid), 12 parts of a surfactant (butyl palmitate) and 30 parts of a solvent (pentanol).

The invention relates to a preparation method of a composition for improving the efficacy of hair growth active substance, including the following steps of, the raw material in parts by weight:
S1, heating and dissolving 10 parts of hair growth active substance (minoxidil sulfate) and 30 parts of pentanol at 90° C. to obtain a first mixture;
S2, heating and melting 25 parts of behenyl alcohol, 50 parts of lanolin, 3 parts of a ginger extract, 15 parts of eicosanoic acid, and 12 parts of butyl palmitate at 90° C. to obtain a second mixture;
S3, adding the first mixture obtained in the S1 step into the second mixture obtained in the S2 step within 4 minutes while stirring at a rotation speed of 400 rpm for 8 minutes, and then homogenizing at a rotation speed of 8000 rpm for 12 minutes to obtain a composition having hair growth active substance.

### Example 12

A composition for improving the efficacy of hair growth active substance comprises the following materials in parts by weight: 10 parts of hair growth active substance (minoxidil and finasteride in a mass ratio of 1:1) dispersed in a carrier, wherein the carrier comprises 100 parts of a penetration enhancer (25 parts of behenyl alcohol, 50 parts of lanolin, 10 parts of a ginger extract, and 15 parts of eicosanoic acid), 20 parts of a surfactant (butyl palmitate) and 30 parts of solvent (pentanol).

The invention relates to a preparation method of a composition for improving the efficacy of hair growth active substance, including the following steps of, the raw material in parts by weight:
S1, heating and dissolving 10 parts of hair growth active substances (minoxidil and finasteride in a mass ratio of 1:1) and 30 parts of pentanol at 100° C. to obtain a first mixture;
S2, heating and melting 25 parts of behenyl alcohol, 50 parts of lanolin, 10 parts of a ginger extract, 15 parts of eicosanoic acid, and 20 parts of butyl palmitate at 100° C. to obtain a second mixture;

S3, adding the first mixture obtained in the S1 step into the second mixture obtained in the S2 step within 5 minutes while stirring for 5 minutes at a rotation speed of 500 rpm, and then homogenizing for 10 minutes at a rotation speed of 10000 rpm to obtain a composition having hair growth active substance.

It will be appreciated that in the above Examples and alternatives thereto, the hair growth active substance may be replaced with at least one of minoxidil or pharmaceutically acceptable salt thereof, finasteride; the alcohol in the penetration enhancer may be replaced with a C₁₀-C₂₅ alcohol; the pharmaceutically acceptable grease can be replaced with at least one of lanolin, white vaseline, and yellow vaseline; the alkanoic acid in the penetration enhancer may be replaced with tetradecanoic acid, hexadecanoic acid or octadecanoic acid; the surfactant may be replaced by C₃-C₆ alcohol esters of C₁₀-C₂₂ fatty acids; the solvent may be replaced with C₂-C₆ monohydric alcohol or C₂-C₆ polyhydric alcohol. The replaced composition also has the technical effects of the present invention.

In the present invention, performance tests were performed using Examples 1, 2, and 8 as examples, and other examples also had the following test performance.

### Performance Test:

### (1) Permeability Test of Isolated Skin

Test materials: skin of depilating mice, commercially available minoxidil tincture, compositions of examples 1, and 8 of the present invention
Test instruments: transdermal tester, high performance liquid chromatography (HPLC) system

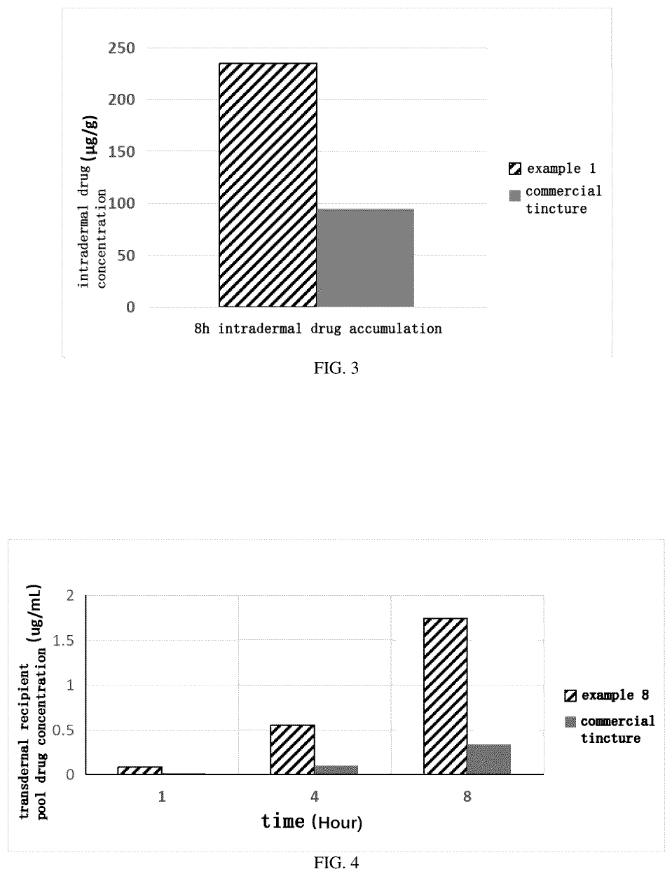

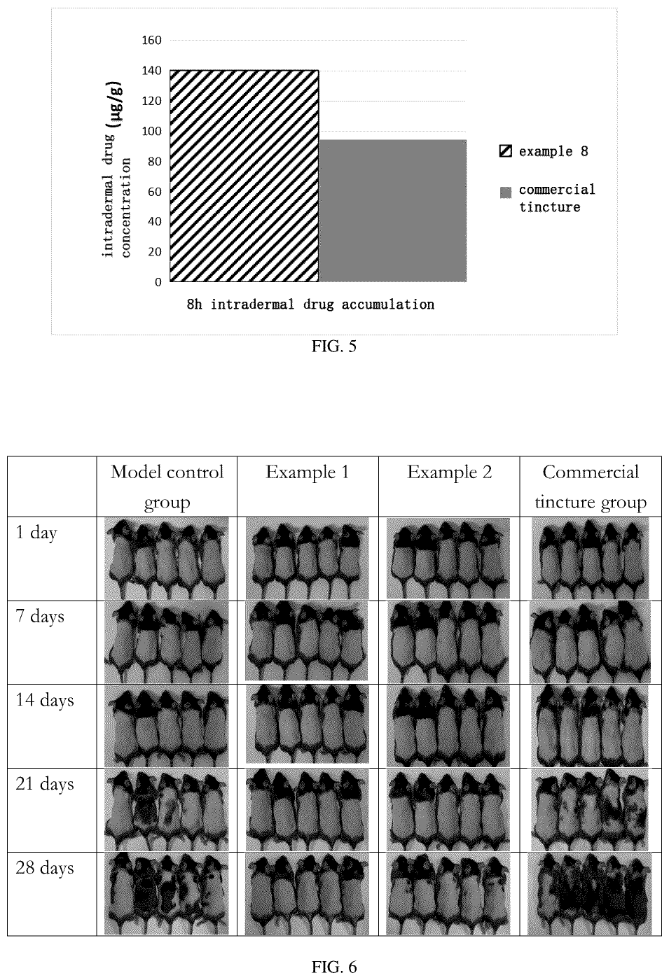

Test description: adopting the method of the widely used transdermal tester (Franz diffusion cell), with a transdermal diffusion bottleneck of area of 1.44 cm² and a recipient volume of 20 mL in the bottle. The skin of depilating mice was placed between the donor and the recipient, and the recipient vial was filled with physiological saline. The system maintained a constant temperature of 32° C. and the recipient vial was magnetically stirred to ensure homogeneity during the test. 0.5 g of the commercially available minoxidil tincture and the composition of examples 1 and 8 of the present invention, respectively, were applied to the skin surface of mice. Three parallel samples per group were collected from the receptor chamber at predetermined time points at the 1^{st}, 4^{th}, 8^{th} hours, respectively; meanwhile, skin samples were collected at the 8th hour, i.e., the skin surface was fully washed with water and the skin was fully cut into pieces, immersed in physiological saline, oscillated and centrifuged to obtain the supernatant. A high performance liquid chromatography (HPLC) system was used to test the content of minoxidil in the recipient fluid at the 1^{st}, 4^{th}, 8^{th} hours and the skin extract supernatant at the 8th hour (i.e., transdermal recipient pool drug concentration and intradermal drug accumulation). The test results are shown in FIGS. 2-5.

Test results showed that the compositions of examples 1 and 8 of the present invention had significantly higher levels of minoxidil in recipient fluids and skin than commercially available minoxidil tinctures, with statistical differences. Therefore, compared with the commercial tincture, the composition for improving the efficacy of hair growth active substance has higher penetration to isolated skin, and the efficacy is obviously improved.

### (2) Animal Test Hair Growth Efficiency Test

Test material: C57BL/6 mice, commercially available 5% minoxidil tincture, compositions of examples 1 and 2 of the present invention

Test description: 18 C57BL/6 mice (male, 6 weeks old) were subjected to back shaving and further hair removal with depilatory cream. After 24 hours, they were randomly divided into model control group (5 mice), example 1 group (5 mice), example 2 group (5 mice), and commercial tincture group (3 mice). Meanwhile, modeling agent application and administration of the treatment were performed for consecutive 21 days. The specific treatments were as follows: for the model control group, 100 μL 0.5% testosterone propionate (prepared with 75% ethanol) was applied to the back of each mouse every day, and no other dosing treatments were given. Examples group and commercial tincture group: every day, 100 μL 0.5% testosterone propionate was applied to the back of each mouse, one hour later, each group was given the first drug treatment (the effective drug dose was 5 mg/time/mouse), and the second drug treatment was conducted at an interval of 6 hours. After the mice were anesthetized on the 1^{st}, 7^{th}, 14^{th} and 21^{st} days of dosing, the hair growth of mice in each group was photographed and recorded. The experimental results are shown in FIG. 6.

The test results showed that the hair rejuvenation rates of mice applying the compositions of example 1 and example 2 of the present invention, respectively, were significantly better than the hair rejuvenation rates of mice applying the commercial tincture group. Therefore, the composition for improving the efficacy of hair growth active substance of the present invention has better hair growth promoting efficacy as compared with commercial tinctures.

The above description is only for the Examples of the present invention and is not intended to limit the patent scope of the present invention. Any equivalent structure or equivalent flow transformation made by using the content of the present specification, or direct or indirect application in other related technical fields, is similarly included in the patent protection scope of the present invention.

What is claimed is:

1. A composition for improving the efficacy of hair growth active substance, consisting of the following materials in parts by weight: 0.5-5 parts of a stabilizer, 20-80 parts of a pharmaceutically acceptable carrier, and 3.1-82 parts of lipophilic particles suspended in the carrier;
the lipophilic particles have core-shell structures or homogeneous entities, and the lipophilic particles comprise 0.5-10 parts of hair growth active substance, 1.6-52 parts of a penetration enhancer, and 1-20 parts of a surfactant;
the penetration enhancer comprises the following materials in parts by weight: 0.5-25 parts of a C₁₀-C₂₅ alcohol, 0.5-5 parts of a pharmaceutically acceptable grease, 0.5-12 parts of a C₁₂-C₂₀ alkanoic acid and 0.1-10 parts of a ginger extract;
the hair growth active substance is at least one of a minoxidil or a pharmaceutically acceptable salt thereof and a finasteride.

2. The composition for improving the efficacy of hair growth active substance according to claim 1, wherein the stabilizer is at least one of a polyethylene glycol, a C₂-C₆ monohydric or polyhydric alcohol, sodium dodecyl sulfate, disodium EDTA, Tween 80, Tween 60, Tween 20, water-soluble cellulose or derivatives thereof, carbomer and xanthan gum.

3. The composition for improving the efficacy of hair growth active substance according to claim 1, wherein the penetration enhancer comprises 0.5-15 parts by weight of a C₁₄-C₂₀ alcohol.

4. The composition for improving the efficacy of hair growth active substance according to claim 1, wherein the pharmaceutically acceptable grease is at least one of lanolin, white vaseline, and yellow vaseline.

5. The composition for improving the efficacy of hair growth active substance according to claim 1, wherein the alkanoic acid in the penetration enhancer is dodecanoic acid, tetradecanoic acid, hexadecanoic acid, heptadecanoic acid, octadecanoic acid or eicosanoic acid.

6. The composition for improving the efficacy of hair growth active substance according to claim 1, wherein the surfactant is a C₃-C₆ alcohol ester of fatty acid; wherein the fatty acid comprises a C₁₀-C₂₂ saturated fatty acid or unsaturated fatty acid; the C₃-C₆ alcohol ester is a monoester or polyester, the weight parts of the C₃-C₆ alcohol ester are 1-15 parts.

7. The composition for improving the efficacy of hair growth active substance according to claim 1, wherein the pharmaceutically acceptable carrier is at least one of water and C₂-C₆ alcohols, wherein the C₂-C₆ alcohols are monohydric alcohols or polyhydric alcohols.

8. A preparation method of a composition for improving the efficacy of hair growth active substance, comprising the steps of, the raw material in parts by weight:
S1, heating and dissolving 0.5-10 parts of hair growth active substance and 20-80 parts of a pharmaceutically acceptable carrier at 60-100° C. to obtain a first mixture, wherein the hair growth active substance is at least one of minoxidil or pharmaceutically acceptable salt thereof and finasteride;
S2, adding 20-60 parts of water, 0.1-10 parts of a penetration enhancer ginger extract and, 0.5-5 parts of a stabilizer into the first mixture in the S1 step, heating and stirring at the temperature of 60-100° C. and the rotating speed of 100-500 rpm to obtain a clarified second mixture;
S3, heating and melting 1.5-42 parts of other penetration enhancer and 1-20 parts of a surfactant at the temperature of 60-100° C. to obtain a third mixture, wherein the other penetration enhancer comprises the following materials in parts by weight: 0.5-25 parts of a C₁₀-C₂₅ alcohol, 0.5-5 parts of a pharmaceutically acceptable grease and 0.5-12 parts of a C₁₂-C₂₀ alkanoic acid;
S4, adding the second mixture obtained in the S2 step into the third mixture obtained in the S3 step within 5 minutes, stirring for 5-15 minutes at the rotating speed of 100-500 rpm, and then homogenizing for 10-20 minutes at the rotating speed of 4000-10000 rpm to obtain the composition that promotes hair growth efficacy with the active substance.

9. A composition for improving the efficacy of hair growth active substance, consisting of the following materials in parts by weight: 0.5-10 parts of hair growth active substance dispersed in a carrier, wherein the carrier comprises 15.1-100 parts of a penetration enhancer, 5-20 parts of a surfactant and 15-30 parts of a solvent;
the penetration enhancer comprises the following materials in parts by weight: 5-25 parts of a C₁₀-C₂₅ alcohol, 5-50 parts of a pharmaceutically acceptable grease, 5-15 parts of a C₁₂-C₂₀ alkanoic acid, and 0.1-10 parts of a ginger extract;
the hair growth active substance is at least one of minoxidil or pharmaceutically acceptable salt thereof and finasteride.

10. The composition for improving the efficacy of hair growth active substance according to claim 9, wherein the pharmaceutically acceptable grease is at least one of lanolin, white vaseline, and yellow vaseline.

11. The composition for improving the efficacy of hair growth active substance according to claim 9, wherein the alkanoic acid in the penetration enhancer is dodecanoic acid, tetradecanoic acid, hexadecanoic acid, heptadecanoic acid, octadecanoic acid or eicosanoic acid.

12. The composition for improving the efficacy of hair growth active substance according to claim 9, wherein the surfactant is a C₃-C₆ alcohol ester of fatty acid; wherein the fatty acid comprises a C₁₀-C₂₂ saturated fatty acid or unsaturated fatty acid; the C₃-C₆ alcohol ester is a monoester or polyester, the weight parts of the C₃-C₆ alcohol ester are 5-12 parts.

13. The composition for improving the efficacy of hair growth active substance according to claim 9, wherein the solvent is a C₂-C₆ monohydric alcohol or a C₂-C₆ polyhydric alcohol.

14. A preparation method of a composition for improving the efficacy of hair growth active substance, comprising the steps of, the raw material in parts by weight:
S1, heating and dissolving 0.5-10 parts of hair growth active substance and 15-30 parts of a solvent at 60-100° C. to obtain a first mixture, wherein the hair growth active substance is at least one of minoxidil or pharmaceutically acceptable salt thereof and finasteride;
S2, weigh a carrier, wherein the carrier comprises 15.1-100 parts of a penetration enhancer, 5-20 parts of a surfactant, and 15-30 parts of a solvent; heating and melting 15.1-100 parts of the penetration enhancer and 5-20 parts of the surfactant at the temperature of 60-100° C. to obtain a second mixture, wherein the penetration enhancer comprises the following materials in parts by weight: 5-25 parts of a C₁₀-C₂₅ alcohol, 5-50 parts of a pharmaceutically acceptable grease, 5-15 parts of a C₁₂-C₂₀ alkanoic acid, and 0.1-10 parts of a ginger extract;
S3, adding the first mixture obtained in the S1 step into the second mixture obtained in the S2 step within 5 minutes, stirring for 5-15 minutes at the rotating speed of 100-500 rpm, and then homogenizing for 10-20 minutes at the rotating speed of 4000-10000 rpm to obtain the composition that promotes hair growth efficacy with the active substance.
